(19) United States
(12) Patent Application Publication
(10) Pub. No.: US 2004/0067595 A1
(43) Pub. Date: Apr. 8, 2004

(54) Assay for s-nitrosothiol compounds

(76) Inventors: Claire A Davies, London (GB); Paul G Winyard, London (GB)

Correspondence Address: DILWORTH PAXSON LLP, 3200 MELLON BANK CENTER, 1735 MARKET STREET, PHILADELPHIA, PA, 19103, US

(21) Appl. No.: 10/362,864

(22) PCT Filed: Aug. 24, 2001
(86) PCT No.: PCT/GB01/03808

(30) Foreign Application Priority Data
Aug. 25, 2000 (GB) 0021051.8
Jun. 12, 2001 (GB) 0114309.8

(51) Int. Cl. G01N 33/00

(52) U.S. Cl. 436/107; 436/173

(57) ABSTRACT

A method for the quantitative measurement of S-nitrosothiols, e.g. S-nitrosoglutathione (RN=57564-91-7), in a biological sample comprises converting the S-nitrosothiols to nitric acid in alkaline solution (pH>10.5), reacting the nitric oxide with a spin trap, e.g. 3,5-dibromo-4-nitrosobenzene sulphonate (DBNBS), or an iron (II) complex of N-methyl-D-glucamine dithiocarbamate (MGD) else diethylcarbamate (DETC), to produce a paramagnetic adduct, and quantifying the paramagnetic adduct using EPR spectrometry. In a second method, a spin trap capable of reacting with the thiyl radical, e.g. 5-(diethoxyphosphoryl)-5-methyl-1-pyrroline-N-oxide 5,5-dimethyl-1-pyrroline-N-oxide, is used. Diagnostic methods based on the quantitative measurement of S-nitrosothiols are also described.

The present invention relates to an assay for the detection of S-nitrosothiols. In particular, the invention relates to an assay method which employs electron paramagnetic resonance (EPR) spectrometry in a quantitative assay for the detection of S-nitrosothiols in a biological sample.

Until relatively recently, nitric oxide (NO) was simply considered to be an environmental pollutant. More recently, however, it has become clear that it is a molecule which is biologically important in both a physiological and a pathological sense. Nitric oxide is implicated in biological processes including control of systemic blood pressure, respiration, digestion, platelet aggregation and cerebral blood flow, as well as contributing to the microbicidal and tumouricidal activities of macrophages and, possibly neutrophils.

However, it is difficult to measure levels of nitric oxide because it exists as a free radical with a short half life of approximately 10 to 30 seconds in aqueous solution and approximately 0.46 ms in whole blood (Feelisch, M, Stampler, J. S., Methods in Nitric Oxide Research, John Wiley & Sons, New York, 1^{st} Edition 49-65 (1996)).

Consequently, many of the reports implicating NO in various physiological processes and many disease states, are based on the measurement of NO metabolites. These metabolites include nitrite, nitrate and S-nitrosothiols. In human blood, NO is oxidised via several intermediates to form the stable end products nitrate and nitrite. These metabolites have been measured in many experiments to implicate NO production.

The present invention, however, relates to the measurement of S-nitrosothiols. S-nitrosothiols are known to be formed in vivo by NO-dependent S-nitrosation of thiol-containing proteins and peptides such as albumin and haemoglobin (Stampler et al, Science, 258(5090), 1898-1902 (1992) and Jia et al, Nature, 380(6571), 221-226 (1996)). The S-nitrosylation of thiols accelerates their oxidation and increases their reactivity in reactions with various functional groups. However S-nitrosothiols are considerably more stable than NO and it therefore appears that S-nitrosothiols may provide a way of regulating the bioavailability of NO and/or serve to increase its range of action. For example, S-nitrosothiols are relied on in the blood for vascular relaxation because free NO cannot co-exist with haemoglobin (Hou et al, Biochem. Biophys. Res. Comm., 228(1), 88-93 (1996)). S-nitrosothiols are also likely to be involved in inflammation via the host defence mechanisms as they have potent antimicrobial properties, whereas NO does not (De Groote et al, P.N.A.S., 92(14), 6399-6403 (1995)). Furthermore, it has been proposed that S-nitrosothiol groups in proteins are important in the metabolism of NO and in the regulation of cellular functions such as the transport and targeting of the NO group to specific thiol regulatory effector sites, including enzymes and signalling proteins. In addition, S-nitrosothiols elicit functions similar to NO¹⁰⁸ such as vasodilation and inhibition of platelet aggregation.

While S-nitrosothiols are hypothesised to be important in the transport and regulation of NO in physiological processes, many questions regarding their mechanism of formation and functional importance still remain. One reason for the lack of information concerning S-nitrosothiols is that, although S-nitrosothiols are more stable than NO¹⁰⁸ , there is at present no simple, sensitive and specific method for the quantitation of S-nitrosothiols in biological samples.

This is reflected in the limited data available on the measurement of S-nitrosothiols in biological fluids. Airway S-nitrosothiols have been measured in patients suffering from severe asthma (Gaston et al, Lancet, 351(9112), 1317-1319 (1998)) and S-nitrosoproteins have been detected in serum and synovial fluid taken from rheumatoid arthritis patients (Hilliquin et al, Arthritis Rheum., 40(8), 1512-1517 (1997)). In the latter case, Hilliquin et al found that the concentration of S-nitrosoproteins in normal control serum was 100-fold higher than the values reported previously (Feelisch & Stampler, (1996) as above).

Some of the methods described for the measurement of S-nitrosothiols in non-biological samples have been used to assay a few biological samples to demonstrate that the method is capable of measuring S-nitrosothiols in biological fluids. For example Marzinzig et al (Nitric Oxide, 1(2), 177-189 (1997)) measured S-nitrosothiols in serum and urine taken from septic shock patients using a method based on the Saville reaction.

At present, the methods available for quantitating the levels of S-nitrosothiols utilise techniques such as spectrophotometry, chemiluminescence, capillary zone electrophoresis and high performance liquid chromatography (Feelisch & Stampler (1996) as above). Generally, these methods lack the sensitivity required for the measurement of S-nitrosothiols in biological samples. The majority of the methods reported do not measure S-nitrosothiols directly but, rather, they measure the products of nitrosothiol decomposition. For example, the Saville reaction decomposes S-nitrosothiols using mercuric ions to give nitronium ions which react with an aromatic amine to produce a highly coloured dye that is measured by spectrophotometry. Chemiluminescence has been used to measure NO released from S-nitrosothiols when they decompose to form NO and thiyl anions according to the equation:

RSNO→RS⁻+NO¹⁰⁸ .

The stability of S-nitrosothiols under physiological conditions is known to be dependent upon various factors including the nature of the thiyl group (RS) to which the NO group is attached. Examples of RS groups include glutathione, cysteine, albumin and haemoglobin residues and, for instance, nitrosocysteine is less stable than nitrosoglutathione and degrades faster. Other factors which affect the stability of S-nitrosothiols include pH, oxygen tension, redox state and the presence of trace amounts of transition metals.

Copper ions are often used to facilitate the breakdown of S-nitrosothiols to yield NO (Fang et al, Biochem. Biophys. Res. Comm., 252(3), 535-540 (1998)). Other methods employ redox systems, alkaline pH and high temperatures to decompose S-nitrosothiols (Samouilov et al, Anal. Biochem., 258, 322-330 (1998)).

The present invention relates to a method of assaying S-nitrosothiols which is quantitative and which is sufficiently sensitive to be used in biological samples.

In the method of the invention, it is particularly important to ensure that the assay is carried out under conditions which do not lead to the decomposition of nitrite. This is because, like S-nitrosothiol, nitrite is also capable of breaking down to form nitric oxide and, of course, it is not possible to distinguish whether nitric oxide is derived from nitrite or from S-nitrosothiols.

Therefore, in a first aspect of the present invention there is provided a method for measuring the concentration of S-nitrosothiol moieties in a sample, the method comprising:

i. Treating a sample with a spin trapping agent capable of reacting with nitric oxide to produce a paramagnetic adduct having a characteristic EPR signal and converting S-nitrosothiol moieties in the sample to nitric oxide; wherein the pH of the sample is adjusted to at least about 10.5 before the conversion of the S-nitrosothiol moieties; and

ii. detecting the presence and quantity of paramagnetic adduct using electron paramagnetic resonance (EPR) spectrometry and calculating from this the concentration of S-nitrosothiol moieties in the sample.

The high pH at which the process is conducted ensures that there is no possibility that any nitrite present in the sample will break down to give nitric oxide. Therefore, there is no possibility that the concentration of nitrite will be determined instead of or in addition to the concentration of S-nitrosothiol.

A further advantage of the high pH at which the process is conducted it that it assists the breakdown of S-nitrosothiols, which are unstable under alkaline conditions, to nitric oxide.

It is not usual to carry out spin trapping reactions and EPR spectrometry at high pH and conventional spin trapping reactions tend to be carried out at about pH 7.4.

EPR spectrometry is a well known spectroscopic technique, although its application to clinical systems is still in its infancy. In EPR spectrometry, a spin trapping agent reacts with a radical of interest to produce a more stable radical adduct that is paramagnetic and hence can be detected by EPR spectrometry. The spin adduct formed can either be measured directly in the sample or extracted into an organic phase before measurement.

EPR spectrometry has been used in combination with spin trapping to measure NO and other radical species (Arroyo et al, Free Radic. Res. Commun., 14, 145-155 (1991) and Pronai et al, Eur. J. Biochem., 202(3), 923-930 (1991)), preferably using a nitroso spin trapping agent such as 3,5-dibromo-4-nitrosobenzene (DBNBS). However, the use of DBNBS is controversial and Pou et al (Biochim. Biophys. Acta, 1201(1), 118-124 (1994)) proposed that the paramagnetic species formed due to the reaction between NO and DBNBS is artifactual.

The controversy concerning the detection of nitric oxide using EPR spectrometry has not been resolved, although Nazhat et al (Biochim. Biophys. Acta, 1427, 276-286 (1999)) did use the technique to detect nitric oxide derived from nitrite.

Nitrite is usually present in biological fluids in a significantly greater quantity than S-nitrosothiols. A number of publications suggest that the concentration of S-nitrosothiols in human blood is in the range of 2×10⁻⁸ to 3×10⁻⁶M (Feelisch and Stampler (1996), Hilliquin et al, (1997)). This would suggest that EPR spectrometry would have insufficient sensitivity to detect S-nitrosothiols since the technique was previously thought to have a limit of detection of about 10⁻⁶M (Feelisch & Stampler (1996); McNamme, “Electron Paramagnetic Resonance Spectroscopy”, in Food analysis. Principles and Techniques. II Physicochemical Techniques, Gruenwedel and Whitaker, Eds., Marchel Dekker Inc., New York, 1984, 343,386; Fischer, “Structure of Free Radicals in ESR Spectroscopy” in Free Radicals, ½, J K Kochi, Ed. From the series Reactive Intermediates in Organic Chemistry, G. A. Olah, Ed., John Wiley and Sons, New York, 1973, 435-491). Surprisingly, we have found that EPR spectrometry can, indeed, be used to detect S-nitrosothiols in blood as well as other human extracellular fluids.

The steps of adding the spin trap and converting S-nitrosothiol to nitric oxide may be carried out in any order, provided that the pH of the sample is adjusted to at least 10.5 before the conversion of the S-nitrosothiol to nitric oxide. Usually, a spin trap solution buffered to at least pH 10.5 is added to the sample and then the sample is treated so as to convert the S-nitrosothiol to nitric oxide.

The step of detecting the presence and quantity of the paramagnetic adduct can be achieved either by subjecting the sample to EPR spectrometry or, alternatively, by extracting the paramagnetic adduct into an organic solvent and subjecting the organic solution to EPR spectrometry. One skilled in the art will be aware that the organic solvent selected will depend upon the nature of the spin trap used and the adduct formed.

Alternatively, for some spin traps it may be advantageous to include the additional step of extracting the S-nitrosothiol moieties into an organic solvent before addition of the spin trap and conversion to nitric oxide and thiyl radicals. This works particularly well for spin traps such as iron (II) complexes of dithiocarbamates, which can complex in organic as well as aqueous solvents. Once again, the organic solvent selected will depend upon the nature of the spin trap used and the adduct formed.

The method of the invention may be improved by the addition to the sample of a compound which reacts with free thiol groups. This effectively removes thiol anion product from the sample mixture so that a reverse reaction in which the S-nitrosothiol is reformed cannot occur. Thus, the reaction equilibrium is moved towards the production of nitric oxide and the amount of free NO available for detection is increased.

Any compound which reacts with free thiol groups may be used, for instance alkylating agents and glycosylating agents. Many different alkylating and glycosylating agents are known and examples of alkylating agents include maleimide, N-alkyl maleimides, N-alkyl phthalimides, iodoacetate, iodoacetamide, iminopyrollidones such as 4-imino-1,3-diazobicyclo-(3,10)-hexan-2-one (Imexon), alkane thiosulphonates, especially methane thiosulphonate, fluoro-substituted alkyl phenols such as 4-trifluoromethyl phenol and erthopeptidyl epoxides. N-C₁-C₄ alkyl derivatives are preferred, and particularly N-methyl, N-ethyl and N-propyl maleimides and phthalimides.

Suitable spin trapping agents for use in the present invention include nitroso compounds such as 3,5-dibromo-4-nitrosobenzene sulphonate (DBNBS), which is converted to a radical product, DBNBS-NO. Derivatives of DBNBS and other nitroso compounds may also be used and these include labelled derivatives such as deuterium labelled DBNBS (DBNBS-d₂), ¹⁵N-labelled DBNBS (DBNBS-¹⁵N) and deuterium and ¹⁵N double labelled DBNBS (DBNBS-d₂—¹⁵N). Alternatively, it is possible to use a nitrosobenzene analogue of DBNBS, which can be labelled with deuterium and/or ¹⁵N in a similar manner to DBNBS. Any other spin trapping agent which can trap NO may also be used, for example nitromethane.

A particularly suitable group of spin trapping agents is iron (II) complexes of dithiocarbarnates such as N-methyl-D-glucamine dithiocarbamate (MGD) or diethyldithiocarbamate (DETC). The iron (II) complexes of these are MGD₂—Fe²⁺ and DETC₂—Fe²⁺ respectively.

These compounds have the advantage that they are more sensitive than many other spin trapping agents and they are therefore particularly well adapted for the quantitative detection of agents, such as S-nitrosothiols, which are present in very low concentrations. In addition, the rate constants for the reactions of dithiocarbamate-iron(II) complexes with nitric oxide are much higher than the rate constants for the reaction of nitric oxide with most other spin trapping agents. For example, the rate constant for the reaction of MGD₂—Fe²⁺ with nitric oxide is 1.28×10⁶M⁻¹s⁻¹. It is important that the rate constant is high as it ensures that the nitric oxide released from the S-nitrosothiol is trapped quickly before it has time to react with other biological oxidants, such as O₂^{•-}and O_{2.}

It is, however, somewhat surprising that the dithiocarbamate complexes work so effectively as it might have been expected that the high pH employed in the process of the present invention would have destabilised the dithiocarbamate-iron (II) complex.

In addition previous workers have experienced problems with the specificity of dithiocarbamate-iron(II) complexes as they react with nitrite to produce a product which is indistinguishable by EPR spectrometry from their product with nitric oxide. Surprisingly, however, the present inventors have had no problems with the specificity of dithiocarbamate-iron (II) complexes, either because of the high pH at which the reaction is conducted or because nitrites were present in the samples only in biological concentrations instead of the high concentrations used by previous investigators. In addition, the present inventors analysed the samples after much less time than in previous experiments and this may also have affected the results. In summary, therefore, the reason for the success of the method of the present invention is not absolutely clear, although there are various possibilities as outlined above. It is clear, however, that the success of the method would not have been predicted from the experience of previous workers in the field.

The spin trapping agent may be present in the reaction mixture in a concentration of from about 10 mM to 500 mM. However, the optimal concentration will depend upon the particular spin trap used.

In general, if nitromethane is used as a spin trap, the concentration will be from about 0.1M to 0.5M, typically about 0.4M.

Nitroso spin traps such as DBNBS are usually used in a concentration of about 0.05 to 0.4M, preferably about 0.2M.

Dithiocarbamates such as MGD and DETC may be used in a concentration of about 15 to 70 mM, but preferably about 25-50 mM.

When using an iron (II) complex of a dithiocarbamate, it is also important to ensure that a molar excess of dithiocarbamate is present and typically, the molar ratio of dithiocarbamate to iron is about 5:1.

When a dithiocarbamate complex is used as the spin trap, the spin trapping reaction must be carried out under anaerobic conditions to prevent the iron (II) from being oxidised to iron (III). Therefore, degassing of the reaction mixture is generally carried out before addition of the dithiocarbamate complex.

Various methods are known for converting the nitrosothiol moieties to nitric oxide and thiyl anions, for example reaction with transition metal ions, particularly copper (I) and copper (II) ions, redox cycling and irradiation. However, the method used will depend upon the spin trapping agent which is to be used.

When the spin trap is a dithiocarbamate such as MGD₂—Fe²⁺ or DETC₂—Fe²⁺, it is not preferable to convert the S-nitrosothiols in the sample to nitric oxide with copper (I) or copper (II) ions. This is because when a dithiocarbamate iron (II) complex reacts with NO, it generates a dithiocarbamate-iron (II)-NO product such as MGD₂—Fe²⁺-NO or DETC₂—Fe²⁺NO. The EPR signal generated by this type of product cannot be separated from the EPR signal generated by copper ions or, indeed, the EPR signals of paramagnetic ions of other transition metals which may be used to convert S-nitrosothiols to nitric oxide.

Therefore, when the spin trap is a dithiocarbamate iron (II) complex, it is preferable to convert the S-nitrosothiols to nitric oxide using a redox cycling system, for example a hydroquinone/quinone system at pH>10.5. This is similar to the system described by Samouilov and Zweier (Anal. Biochem., 258, 322-330 (1998)), although the present inventors have found that heating the sample is not necessary as dithiocarbamates such as MGD appear to attenuate the breakdown of S-nitrosothiols.

The hydroquinone/quinone system is a redox cycling system which breaks down nitrosothiols by altering the redox state. Oxidation of hydroquinone requires two electrons but semiquinone radicals can be formed by a single electron transfer. In addition, a mixture of hydroquinone and quinone forms quinhydrone. This is a charge transfer complex where the hydroquinone acts as the electron donor and the quinone as an electron acceptor. This can also assist the breakdown of nitrosothiols.

It might have been expected that a redox cycling system such as the hydroquinone/quinone system would interfere with the oxidation state of the iron in a dithiocarbamate-iron (II) complex and that this would prevent the complex from trapping the nitric oxide. However, surprisingly, it appears that this is not the case and that the redox cycling system assists the breakdown of S-nitrosothiols without affecting the spin trap.

Conversely, when the spin trap is a nitroso compound such as DBNBS, the quinone/hydroquinone system is not suitable for decomposing the nitrosothiol moieties in the sample because the semiquinone radicals produced by the system interfere with the EPR signal from the DBNBS-NO product (or nitroso spin trap-NO product).

Therefore, for nitroso spin traps, it is preferred to treat the sample with transition metal ions in order to generate NO. Any transition metal may be used but copper ions are commonly used and copper (I) ions have been found to be particularly effective. The breakdown of S-nitrosothiols proceeds via the following reaction.

RSNO+Cu⁺+H⁺→RSH+NO+Cu²⁺

As mentioned above, the conversion of nitrosothiols to nitric oxide can also be achieved by adjusting the pH or oxygen tension of the system. The process of the present invention is, as mentioned above, carried out at a pH of at least 10.5 and, since S-nitrosothiols are unstable under alkaline conditions, this will assist their decomposition. Also, since the conversion of S-nitrosothiols to nitric oxide is a reduction reaction, a decrease in the amount of oxygen present in the reaction mixture will lead to increased production of NO. This is particularly significant when the spin trapping agent is a dithiocarbamate-iron (II) complex since spin trapping with these agents is, as discussed above, carried out under anaerobic conditions.

A further method of converting the S-nitrosothiol to nitric oxide is by irradiation (Arnelle et al, Nitric Oxide, 1(1), 56-64 (1997)), a method which can be used for any of the spin traps mentioned above. In this method, a thiyl radical is produced instead of a thiyl anion and this radical be trapped instead of or in addition to the NO using a spin trapping agent which gives a characteristic signal with thiyl radicals. If the right spin trap is chosen, the paramagnetic species will give an EPR signal which will distinguish between different thiyl radicals. For example, different signals will be obtained for cysteine, albumin, haemoglobin and glutathione radicals. Therefore, this method can be used in addition to or instead of trapping nitric oxide in order to distinguish and quantify different S-nitrosothiols present in the sample.

Thus, in a further aspect of the invention, there is provided a method for measuring the concentration of different S-nitrosothiol moieties in a sample, the method comprising the steps of:

i. Treating a sample with a spin trapping agent capable of reacting with a thiyl radical to produce a paramagnetic adduct which has a characteristic EPR signal and irradiating the sample to convert S-nitrosothiol moieties in the sample to nitric oxide and thiyl radicals; and

ii. detecting the presence and quantity of paramagnetic adduct using electron paramagnetic resonance (EPR) spectrometry and calculating from this the concentration of S-nitrosothiol moieties in the sample.

Particularly suitable spin traps for thiyl radicals include 5-(diethoxyphosphoryl)-5-methyl-1-pyrroline-N-oxide and 5,5-dimethyl-1-pyrroline-N-oxide.

As with the first aspect of the invention, the step of detecting the presence and quantity of the paramagnetic adduct can be achieved either by subjecting the sample to EPR spectrometry or, alternatively, by extracting the paramagnetic adduct into an organic solvent and subjecting the organic solution to EPR spectrometry. One skilled in the art will be aware that the organic solvent selected will depend upon the nature of the spin trap used and the adduct formed.

Alternatively, as was also described for the first aspect of the invention, it may be advantageous with some spin traps to include the additional step of extracting the S-nitrosothiol moieties into an organic solvent before addition of the spin trap and conversion to nitric oxide and thiyl radicals.

An alternative method for measuring the concentrations of individual species of nitrosothiol is by combining the method of the first aspect of the invention with an additional separation technique, for example a chromatographic method such as HPLC or an electrophoretic method such as capillary electrophoresis. Such methods could be carried out either before or after adding the spin trap.

The methods of the present invention can be used to assess the nitrosothiol content of any type of chemical or biological fluid but are particularly suitable for use with biological samples where the concentration of S-nitrosothiols is low and cannot be measured quantitatively by known methods. The methods of the present invention can quantifiably detect S-nitrosothiols at concentrations as low as 10⁻⁸M.

Thus, the methods can be used to determine the concentration of S-nitrosothiols in, for example, human or animal whole blood, serum, plasma, synovial fluid, urine cerebrospinal fluid, peritoneal fluid, gingival crevicular fluid or any other tissue or extracellular fluid of human or animal origin. Alternatively, the concentration of S-nitrosothiols in other biological media, such as cell culture media, or in a chemical system, may be determined.

When the sample to be tested is a blood product, it is preferable to add a chelating agent such as ethylene diamine tetraacetic acid (EDTA) to the sample on or soon after collection in order to chelate excess transition metal ions which could react with S-nitrosothiols during sample preparation and storage.

Since nitrosothiol levels are affected in patients suffering a wide variety of diseases and conditions, the method of the invention may be useful in diagnosing these conditions, determining suitable treatment and monitoring the progression of disease and the effectiveness of treatment.

Diseases and conditions in which nitrosothiol levels are affected include septic shock, renal disease, cardiovascular disease, asthma, rheumatoid arthritis, systemic microbial infections such as tuberculosis, diabetes, inflammatory joint diseases, cerebral ischaemia and many others.

In addition, some drugs, such as the organic nitrates used in the treatment of angina, generate NO and may thereby affect S-nitrosothiol levels. Therefore, the method of the invention may also be useful in monitoring the efficacy of such therapies, particularly in cases of “nitrate tolerance”. In addition, certain drugs under development, including nitrosylated non steroidal antiinflammatory drugs (NSAIDs), contain a nitrosothiol group and the process of the invention may be used in evaluating the metabolism and gastrointestinal toxicity of such drugs.

Therefore, in a further aspect of the invention, there is provided a method of diagnosing or monitoring in a patient the progress or treatment of a disease or condition in which S-nitrosothiol levels are affected, or of monitoring or evaluating the efficacy or toxicity of a drug therapy which affects S-nitrosothiol levels, the method comprising:

i. Treating a sample of body fluid from the patient with a spin trapping agent capable of reacting with nitric oxide to produce a paramagnetic adduct which has a characteristic EPR signal and converting S-nitrosothiol moieties in the sample to nitric oxide; wherein the pH of the sample is adjusted to at least about 10.5 before the conversion of the S-nitrosothiol moieties; and

ii. detecting the presence and quantity of paramagnetic adduct using electron paramagnetic resonance (EPR) spectrometry and calculating from this the concentration of S-nitrosothiol moieties in the sample.

The sample may be whole blood, serum, plasma, synovial fluid or urine collected from the patient.

Preferred conditions are as set out for the first aspect of the invention.

The invention will now be described in greater detail with reference to the following non-limiting examples and the drawings in which:

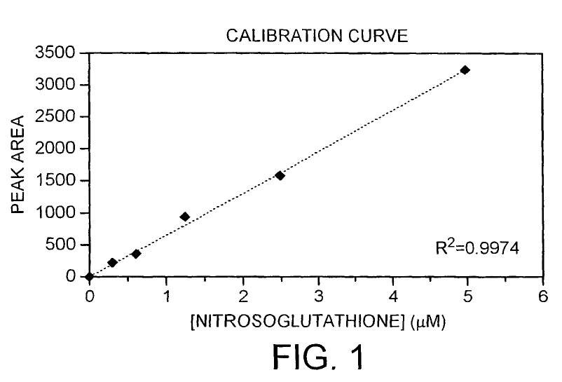

FIG. 1 is a calibration curve showing nitrosoglutathione concentration vs. the peak area of the (MGD)₂—Fe²⁺—NO signal detected by EPR spectrometry. The regression coefficient indicates that the experimental method is linear.

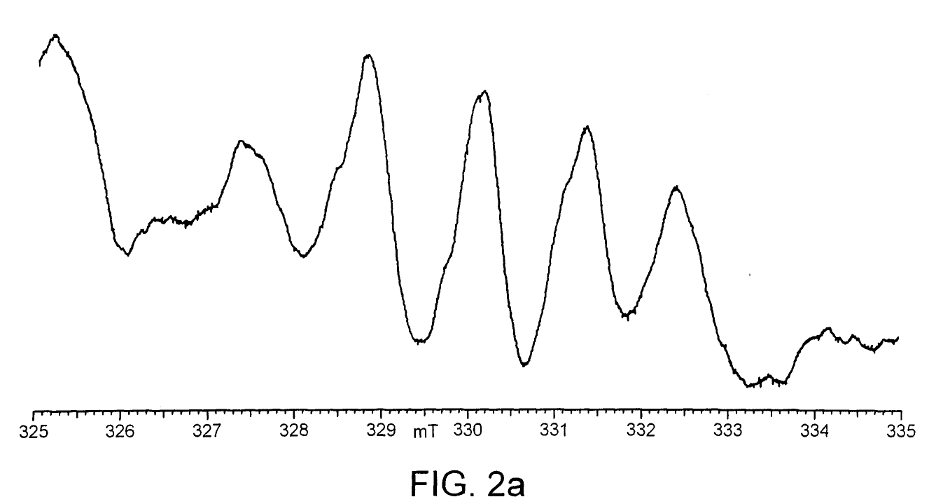

FIG. 2 is a set of spectra demonstrating the measurement of NO released from nitrosothiols using (MGD)₂—Fe²⁺ and 0.01M 1,4-benzoquinone/0.1M hydroquinone in Tris-HCl at pH>10.5 a) serum taken at a receiver gain of 10000 and b) synovial fluid taken at a receiver gain of 5000. The instrument parameters were: microwave frequency 9.45 GHz, microwave power 20 mW, centre field 330.0 mT, sweep width ±5 mT sweep time 120 s, number of data points 8192, time constant ls, modulation frequency 100 kHz, modulation width 0.4 mT.

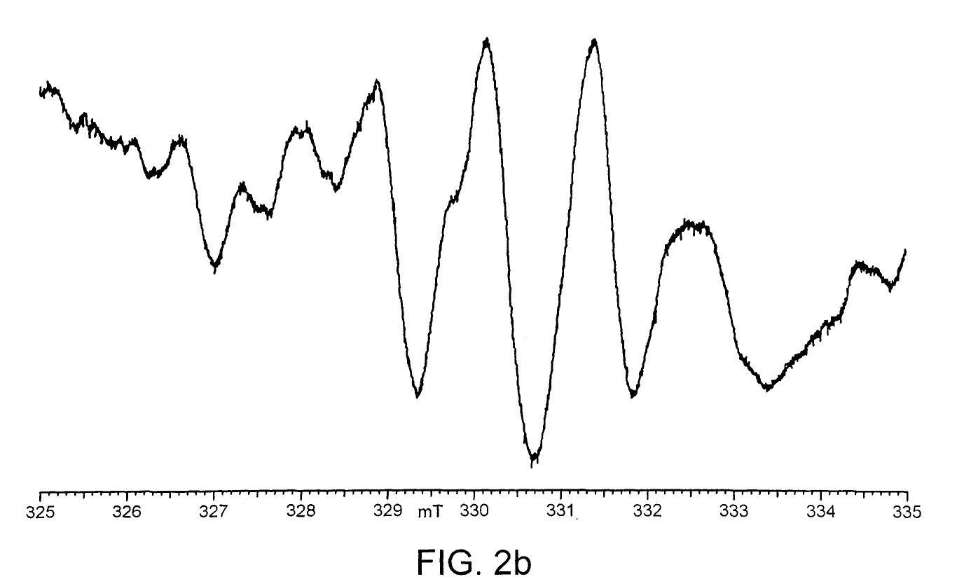

FIG. 3 illustrates the proposed mechanism for S-nitrosothiol decomposition by DETC (taken from Arnelle et al, Nitric Oxide, 1(1), 56-64 (1997)).

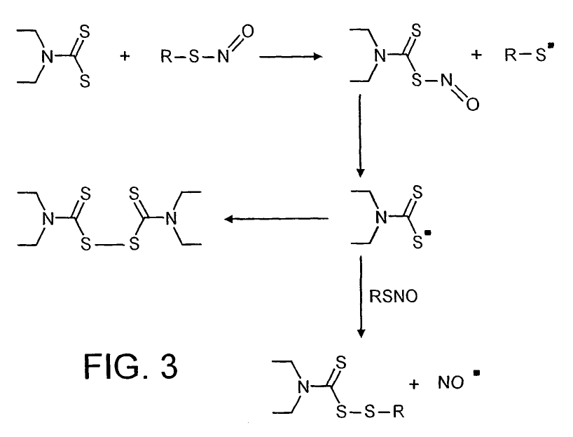

FIG. 4 shows the measurement of NO released from nitrosoglutathione (0.5 mM) using DBNBS prepared in Tris-HCl at pH>10.5 with 100 μM Cu(II)SO₄. The EPR spectra were taken 29 hours after the components had been incorporated. Signal (a) corresponds to the DBNBS-SO₃^{—}adduct and signal (b) corresponds to the DBNBS-NO product. The instrument parameters were:-microwave frequency 9.45 Ghz, microwave power 10 mW, centre field 336.0 mT, sweep width ±5 mT, sweep time 150 s, number of data points 8192, time constant 0.3 s, modulation frequency 100 kHz, reciever gain 500, modulation width 0.2 mT.

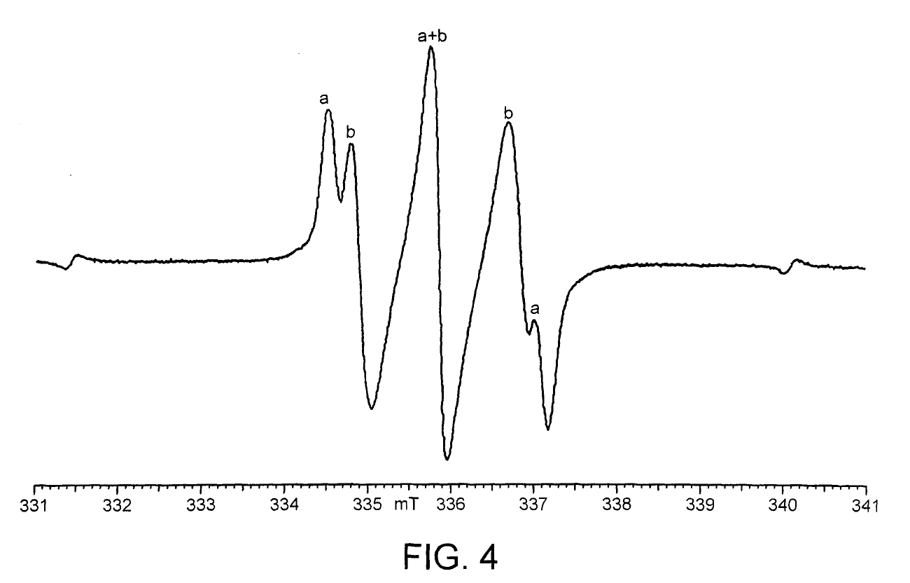

FIG. 5 shows the effect of N-ethylmaleimide (NEM) on the (MGD)₂—Fe²⁺—NO¹⁰⁸ signal height measured in the RSNO's assay. The NO^{•}released from RSNO's was measured in the presence and absence of NEM in a 1.25 mM nitrosoglutathione aqueous standard solution (A) and a RA synovial fluid sample spiked with 1000 nM nitrosoglutathione (B). The duplicate values varied by less than 10%.

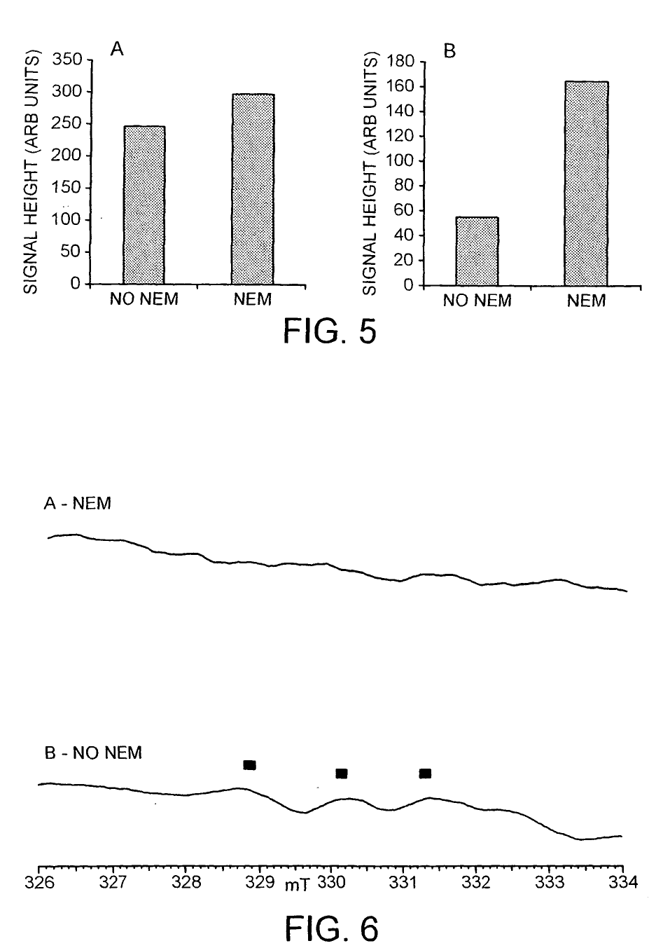

FIG. 6 illustrates the effect of NEM on the (MGD)₂—Fe²⁺—NO^{•}signals sometimes seen in(MGD)₂—Fe²⁺ preparations. Spectrum A represents the EPR spectrum from a solution containing 50 mM (MGD)₂—Fe²⁺(50 mM MGD, 10 mM Fe^{2+;} 500 μL), 250 mM NEM (10 μL) and deionised water (490 μL). Spectrum B was the same as spectrum A except that the NEM was substituted with deionised water. The instrument parameters were: microwave frequency 9.45 GHz, microwave power 20 mW, centre field 330.0 mT, sweep width ±4 mT, sweep time 80 s, number of data points 8192, time constants 1 s, modulation width 1 mT, receiver gain 10000. Both EPR spectra were taken by averaging 5 scans. The three solid squares in FIG. 6B indicate the postion of the three line signal assigned to (MGD)₂Fe²⁺NO^{•}.

### EXAMPLE 1

Blood and synovial fluid samples were taken from patients suffering with rheumatoid arthritis (RA). All samples were taken in plain glass tubes and then centrifuged at 3000 rpm for 5 mins. Serum and synovial fluid were removed and stored at −70° C. until analysis.

A solution of 0.01M 1,4- benzoquinone and 0.1M hydroquinone was prepared in TrisHCl buffer that had been adjusted to pH>10.5. A solution of Tris-HCl adjusted to pH >10.5 was degassed for 15 mins using nitrogen gas. The degassed buffer was then used to prepare a 10 mM ammonium ferrous sulphate solution. The ammonium ferrous sulphate solution was then added to MGD powder in a vacutainer to form the MGD₂—Fe²⁺ complex (final concentration of MGD was 50 mM). MGD_{2—}Fe²⁺ solution (100μL) was then removed using a gas tight syringe and added to 100 μL hydroquinone/quinone system in a vacutainer. Nitrosoglutathione (300 μL) (5 μM final concentration) solution was then added. In order to construct a calibration curve the concentration of nitrosoglutathione was varied to give final concentrations of 2.5, 1.25, 0.6125 and 0.3125 μM. Synovial fluid or serum (300 μL) from a rheumatoid arthritis (RA) patient was added to the system instead of the nitrosoglutathione. All samples were tested by EPR spectrometry approximately 5 mins after the nitrosoglutathione or the sample had been added.

The EPR spectra were obtained from a JEOL JES-RE1X spectrometer (Jeol (UK) Ltd, Welywn Garden City, England) equipped with an ES-UCX2 cylindrical mode X-band cavity. Samples were analysed at room temperature in a WG-LC-11 quartz flat cell (Wilmad Glass, Buena, N.J.). The instrument parameters were: microwave frequency 9.45 GHz, microwave power 20 mW, center field 330.0 mT, sweep width ±5 mT, sweep time 250 s, number of data points 8192, time constant 1 s, modulation frequency 100 kHz, receiver gain 2-10000, modulation width 0.4 mT.

The nitrosoglutathione added to the system was decomposed and the spin trap reacted with the NO liberated to give the paramagnetic complex, MGD₂—Fe²⁺—NO. The characteristic 3line signal of MGD₂—Fe²⁺—NO was detected by EPR spectrometry. A calibration curve was constructed by plotting the peak area of the MGD₂—Fe²⁺—NO signal against the concentration of nitrosoglutathione added to the system. A linear regression coefficient of 0.9974 was calculated (FIG. 1). S-nitrosothiols (RSNO's) present in the serum and synovial fluid samples were decomposed and the NO released was measured in the form of MGD₂—Fe²⁺—NO (FIG. 2a and b).

In this example the hydroquinone/quinone system was used at pH>10.5 to decompose the RSNOs present. Samouilov and Zweier (1998) demonstrated that they could facilitate the decomposition of RSNOs within 10-30 s if they used this system and also heated their samples to 60° C. Our experiments suggest that heating the sample is not necessary as it seems that MGD attenuates the breakdown of the RSNOs by the mechanism shown in FIG. 2. Although MGD₂—Fe²⁺ has not yet been shown to break down RSNOs, Arnelle et al (1997) have shown that diethyldithiocarbamate (DETC) (another diothiocarbamate) will facilitate their decomposition by this mechanism. Metal ions such as copper are usually used to decompose RSNOs. Unfortunately, they cannot be used here as copper (II) is paramagnetic and its EPR signal cannot be separated from the MGD₂—Fe²⁺—NO EPR signal.

### EXAMPLE 2

DBNBS solution (0.2M) was prepared in Tris-HCl adjusted to pH>10.5. The DBNBS solution was added to the nitroso-glutathione to give a final concentration of 0.1M DBNBS and 0.5 mM nitrosoglutathione. Copper sulphate (to give a final concentration of 100 μM) was then added to the reaction mixture. The mixture was tested by EPR spectrometry, immediately and then after 24 hours.

The EPR spectra were obtained from a JEOL JES-RE1X spectrometer (Jeol (UK) Ltd, Welywn Garden City, England) equipped with an ES-UCX2 cylindrical mode X-band cavity. Samples were analysed at room temperature in a WG-LC-11 quartz flat cell (Wilmad Glass, Buena, N.J.). The instrument parameters were: microwave frequency 9.45 GHz, microwave power 4 mW, centre field 336.0 mT, sweep width ±5 mT, sweep time 120 s, number of data points 8192, time constant 0.1 s, modulation frequency 100 kHz, receiver gain 1600-10000, modulation width 0.079 mT.

The nitrosoglutathione added to the system was decomposed and the spin trap reacted with the NO liberated to give the paramagnetic product, DBNBS-NO. The characteristic 3-line signal of DBNBS-NO was detected by EPR spectrometry. When the samples were retested after 29 hours, the DBNBS-NO signal had increased in height when compared to the signal height measured initially (FIG. 4). The samples were retested after 29 hours as the accumulation of the DBNBS-NO product is thought to be greatest at this point.

In this example DBNBS is used as the spin trap and copper (II) ions are used along with alkaline pH to decompose the RSNOs. This is possible as the DBNBS-NO product has a large g-value and the signals do not overlap. The hydroquinone/quinone system cannot be used with DBNBS as the semiquinone radicals produced by the system interfere with the EPR signal from the DBNBS-NO product. Alkaline conditions were used in all cases as the stability of RSNOs is greatly reduced at high pH.

### EXAMPLE 3—PRE-INCUBATION OF SAMPLE WITH ALKYLATING AGENT

In order to improve the results of the assay, biological fluids (700 μL) were incubated with 10 μL of 250 mM N-ethylmaleimide (NEM) for 20 mins before the addition of the spin trap and decomposition mixture. NEM is known to alkylate free thiol groups (Mead et al, Hypertension, 34(6), 1275-1280 (1999)), thereby preventing the NO^{•}released from RSNO's from reforming RSNO's. Consequently, the amount of free NO^{•}available to react with the spin trap is increased. The following three experiments were performed:

1) 250 mM NEM (10 μL) was added to 500 μL deionised water;

2) 250 mM NEM (10 μL) was added to 500 μL of 1.25 mM nitrosoglutathione solution; and

3) 250 mM NEM (10 μL) was added to 500 μL RA synovial fluid, which had been spiked with 1000 nM nitrosoglutathione.

In experiments 1-3, the samples were mixed with 500 μL 50 mM (MGD)₂—Fe²⁺ (50 mM MGD, 10 mM Fe²⁺) prepared in 0.2M CAPS buffer and tested by EPR spectrometry. Each experiment was repeated with NEM being substituted with deionised water. All experiments were performed in duplicate and the results are presented in FIGS. 5 and 6.

As can be seen from FIG. 5, the presence of NEM caused an increase in the signal height of the (MGD)₂—Fe²⁺—NO¹⁰⁸ signal detected when the RSNO's present in the standard solution (2) and the spiked synovial fluid sample (3) were measured. In the synovial fluid sample, the effect of NEM was more pronounced, probably due to the fact that a large number of protein thiols such as albumin were present. Importantly these results also showed that the NEM did not destabilise the (MGD)₂—Fe²⁺ complex by alkylating the sulphur atoms, which react with the iron ions to form the spin trapping complex. Experiment 1 demonstrated that NEM can prevent the generation of the small (MGD)₂—Fe²⁺—NO^{•}signal sometimes seen in blank samples (FIG. 6). When a 250 mM NEM solution was analysed a small broad singlet was present in the EPR spectrum, which overlapped with the first line of the (MGD)₂—Fe²⁺—NO^{•}signal. In order to allow for this, all data analysis was performed from then onwards on the middle line of the EPR signal.

Finally, the assay precision was repeated as alterations had been made to the method. A synovial fluid sample was spiked with 1000 nM nitrosoglutathione. The sample (0.7 mL) was then incubated with 250 mM NEM (10 μL) for 20 mins before analysis. The sample (100 μL) was added to 100 μL 50 mM (MGD)₂—Fe²⁺ (50 mM MGD, 10 mM Fe²⁺) prepared in 0.2M CAPS buffer at pH 10.5. Five samples were prepared in this way and then analysed by EPR spectrometry. The %RSD was calculated to be 4.65%.

### REFERENCES

Arnelle, D. R., Day, B. J. and Stampler, J. S. (1997) Nitric Oxide 1 (1) 56-64.

Arroyo CM. Kohno M (1991) Free Rad Res Comm. 14(2):145-55.

De Groote MA. Granger D. Xu Y. Campbell G. Prince R. Fang FC (1995). PNAS. 92(14):6399-403.

Fang K. Ragsdale NV. Carey RM. MacDonald T. Gaston B (1998). Biochem Biophys Res Comm. 252(3):535-40.

Feelisch, M. Stampler, J. S. John Wiley & Sons, New York 1st Edition. 1996, pp 49-65.

Gaston B. Sears S. Woods J. Hunt J. Ponaman M. McMahon T. Stamler JS (1998). Lancet. 351(9112):1317-9.

Hilliquin, P., Bordiere, D., Hemvann, A., Menkes, C. J. and Ekindjan, O. G. (1997). Artlritis Rheumi 40 (8) 1512-7.

Hou Y. Guo Z. Li J. Wang PG. (1996). Biochem Biophys Res Comm. 228(1):88-93.

Ichimori K. Arroyo CM. Nakazawa H. (1996) Method Enz. 268:203-11.

Jia L. Bonaventura C. Bonaventura J. Stamler JS (1996). Nature. 380(6571):221-6.

Marzinzig M. Nussler AK. Stadler J. Marzinzig E. Barthlen W. Nussler NC. Beger HG. Morris SM Jr. Bruckner UB (1997). Nitric Oxide. 1(2):177-89.

Nazhat, N., Saadalla-Nazhat, R. A., Fairbum, K., Jones, P., Blake D. R., Nielsen, B. R., Symons, M. R. C., Winyard, P. G (1999). Biochim Biophys Acta. 1427:276-286.

Pronai L. Ichimori K. Nozaki H. Nakazawa H. Okino H. Carmichael AJ. Arroyo CM. (1991). Eur J Biochem. 202(3):923-30.

Pou S. Keaton L. Surichamom W. Frigillana P. Rosen GM. (1994) Biochim Biophys Acta. 1201(1):118-24.

Samouilov, A. and Zweier, J. L. (1998). Anzal Biochem. 258, 322-30.

Stamler JS. Singel DJ. Loscalzo J (1992). Science. 258(5090):1898-902.

1. A method for measuring the concentration of S-nitrosothiol moieties in a sample, the method comprising the steps of:
i. Treating a sample with a spin trapping agent capable of reacting with nitric oxide to produce a paramagnetic adduct which has a characteristic EPR signal and converting S-nitrosothiol moieties in the sample to nitric oxide; wherein the pH of the sample is adjusted to at least about 10.5 before the conversion of the S-nitrosothiol moieties and wherein no heating of the sample takes place; and
ii. detecting the presence and quantity of paramagnetic adduct using electron paramagnetic resonance (EPR) spectrometry and calculating from this the concentration of S-nitrosothiol moieties in the sample.

2. A method of diagnosing or monitoring in a patient the progress or treatment of a disease or condition in which S-nitrosothiol levels are affected, or of monitoring or evaluating the efficacy or toxicity of a drug therapy which affects S-nitrosothiol levels, the method comprising:
i. Treating a sample of body fluid from the patient with a spin trapping agent capable of reacting with nitric oxide to produce a paramagnetic adduct which has a characteristic EPR signal and converting S-nitrosothiol moieties in the sample to nitric oxide; wherein the pH of the sample is adjusted to at least about 10.5 before the conversion of the S-nitrosothiol moieties and wherein no heating of the sample takes place; and
ii. detecting the presence and quantity of paramagnetic adduct using electron paramagnetic resonance (EPR) spectrometry and calculating from this the concentration of S-nitrosothiol moieties in the sample.

3. A method as claimed in claim 1 or claim 2 wherein the paramagnetic adduct is extracted into an organic solvent before being detected by EPR spectrometry.

4. A method as claimed in claim 1 or claim 2, further comprising the additional step of extracting the S-nitrosothiol moieties into an organic solvent before their conversion to nitric oxide.

5. A method as claimed in any one of claims 1 to 4, further comprising the step of adding to the sample of a compound which reacts with free thiol groups and prevents S-nitrosothiol compounds from reforming.

6. A method as claimed any one of claims 1 to 5, wherein the spin trapping agent is a nitroso compound, nitromethane or an analogue or derivative of any of these.

7. A method as claimed in claim 6, wherein the spin trapping agent is:
3,5-dibromo-4-nitrosobenzene sulphonate (DBNBS);
an analogue or derivative of any of the above.

8. A method as claimed in claim 7, wherein the analogue is a deuterium labelled, ¹⁵N-labelled or ¹⁵N and deuterium double labelled derivative.

9. A method as claimed in any preceding claim wherein the spin trapping agent is a nitromethane and is present in the reaction mixture in a concentration of about 0.4M.

10. A method as claimed in any one of claims 1 to 8 wherein the spin trapping agent is a nitroso compound and is present in the reaction mixture in a concentration of about 0.2M.

11. A method as claimed in any one of claims 6 to 10 wherein the S-nitrosothiol is converted to nitric oxide by treatment with transition metal ions.

12. A method as claimed in claim 11, wherein the transition metal ions are copper (I) or copper (II) ions.

13. A method as claimed in any one of claims 1 to 5 where spin trapping agent is an iron (II) complex of a dithiocarbamate.

14. A method as claimed in claim 13, wherein the spin trapping agent is an iron (II) complex of N-methyl-D-glucamine dithiocarbamate (MGD) or an iron (II) complex of diethyldithiocarbamate (DETC).

15. A method as claimed in claim 13 or claim 14 wherein the spin trapping agent is present in the reaction mixture in a concentration of about 25-50 mM.

16. A method as claimed in any one of claims 13 to 15 wherein the spin trapping reaction is carried out under anaerobic conditions.

17. A method as claimed in any one of claims 13 to 16, wherein the S-nitrosothiol is converted to nitric oxide via a redox cycling system.

18. A method as claimed in claim 17, wherein the redox cycling system is the hydroquinone/quinone system.

19. A method as claimed in any one of claims 1 to 10 or 13 to 18 (when not dependent on claims 11 and 12) wherein the S-nitrosothiol is converted to nitric oxide by irradiation.

20. A method as claimed in claim 17 or claim 18, further including the step of trapping thiyl radicals with a spin trapping agent, which gives a characteristic signal for individual thiyl radicals.

21. A method as claimed in any preceding claim wherein the sample is human or animal whole blood, serum, plasma, synovial fluid, urine, cerebrospinal fluid, peritoneal fluid, gingival crevicular fluid or any other tissue or extracellular fluid, or a cell culture medium or a chemical system.

22. A method as claimed in claim 21 wherein the sample is a blood product and a chelating agent is added to the sample on or soon after collection.
